(19) United States
(12) Patent Application Publication
(10) Pub. No.: US 2009/0247870 A1
(43) Pub. Date: Oct. 1, 2009

(54) ULTRASONIC DIAGNOSTIC APPARATUS

(76) Inventors: Shinichi Amemiya, Tokyo (JP); Dai Morita, Tokyo (JP); Koji Yanagihara, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/410,802

(22) Filed: Mar. 25, 2009

(30) Foreign Application Priority Data
Mar. 28, 2008 (JP) 2008-087128

(51) Int. Cl. A61B 8/00

(52) U.S. Cl. 600/437

(57) ABSTRACT

An ultrasonic diagnostic apparatus includes an operating device for an operator to input an instruction, an image display device for displaying an ultrasonic image, and a processing device that generates the ultrasonic image based on a reception signal resulting from transmission of an ultrasonic wave for display on the display device. The image display device is mounted removably through an arm, which has a movable portion and extends up to near a table for resting a subject thereon, to any of a ceiling, a wall, and a floor surface of an installation room, or to an image display device mounting member fixed within the installation room. The operating device is mounted removably to an operating device mounting member arranged near the table. The processing device is mounted removably to a processing device mounting member arranged near the table.

### CROSS REFERENCE TO RELATED APPLICATIONS

This application claims the benefit of Japanese Patent Application No. 2008-087128 filed Mar. 28, 2008, which is hereby incorporated by reference in its entirety.

### BACKGROUND OF THE INVENTION

The embodiments described herein relate to an ultrasonic diagnostic apparatus that generates an ultrasonic image on the basis of an echo signal resulting from transmission of an ultrasonic wave.

According to the configuration of a conventional ultrasonic diagnostic apparatus, an operating device for an operator to input an instruction, an image display device for the display of an ultrasonic image, and a processing device for making control to generate ultrasonic image data on the basis of an echo signal resulting from transmission of an ultrasonic wave and display the data, are movable all together (see, for example, Japanese Unexamined Patent Publication No. 2003-339708).

An ultrasonic diagnostic apparatus is relatively heavy and is not easily movable and therefore it actually is often used while being placed at a specific position without movement. Even when it is moved, the frequency of movement may be low. Such being the case, a fixed type ultrasonic diagnostic apparatus is now under study.

However, even in the fixed type, there is no denying the possibility of using the ultrasonic diagnostic apparatus in another place. Accordingly, it is also desired that the ultrasonic diagnostic apparatus be employable also in another place.

On the other hand, there are various inspection sites, including neck, abdomen and legs. It is desired that the operating device and the image display device be easily movable to a position permitting easy inspection by an inspector. Particularly, in the case of a fixed type ultrasonic diagnostic apparatus, it is necessary that the operating device and the image display device be movable.

It is desirable that the problem described previously is solved.

### BRIEF DESCRIPTION OF THE INVENTION

A first aspect provides an ultrasonic diagnostic apparatus including: an operating device for an operator to input an instruction; an image display device for the display of an ultrasonic image; and a processing device for making control to generate an ultrasonic image on the basis of a reception signal resulting from transmission of an ultrasonic wave and display the ultrasonic image on the display device, wherein: the image display device is amounted removably through an arm which has a movable portion and extends up to near a table for resting a subject thereon, to any of a ceiling, a wall and a floor surface of an installation room with the table installed therein, or to an image display device mounting member fixed within the installation room; the operating device is mounted removably to an operating device mounting member arranged near the table; and the processing device is mounted removably to a processing device mounting member arranged near the table.

A second aspect provides an ultrasonic diagnostic apparatus including: an operating device for an operator to input an instruction; an image display device for the display of an ultrasonic image; and a processing device for making control to generate an ultrasonic image on the basis of a reception signal resulting from transmission of an ultrasonic wave and display the ultrasonic image on the image display device, wherein: the operating device being mounted removably through an arm, which has a movable portion and extends up to near a table for resting a subject thereon, to any of a ceiling, a wall and a floor surface of an installation room with the table installed therein, or to an operating device mounting member fixed within the installation room; the image display device is mounted removably to an image display device mounting member arranged near the table; and the processing device is mounted removably to a processing device mounting member arranged near the table.

A third aspect in combination with the above first or second aspect provides an ultrasonic diagnostic apparatus wherein: the image display device mounting member, the operating device mounting member, or the processing device mounting member, is a columnar member capable of extension and retraction; and the columnar member is fixed within the installation room by being adjusted to a length of abutment in a pressed state against the ceiling or the floor surface of the installation room.

A fourth aspect in combination with any of the above first to third aspects provides an ultrasonic diagnostic apparatus wherein at least one of the image display device and the processing device is arranged on the side opposite to the arranged side of the operating device with respect to the table.

A fifth aspect in combination with any of the above first to fourth aspects provides an ultrasonic diagnostic apparatus wherein: the processing device mounting member has a mounting portion having a processing device mounting surface perpendicular to the floor surface, the mounting portion having a retention hole and with a connector for connection thereto of the processing device being provided so as to be movable in a direction perpendicular to the floor surface; the processing device has a retention lug for engagement in the retention hole on a surface thereof which confronts the processing device mounting surface when mounted to the processing device mounting member; and the retention hole has a shape and size permitting the retention lug to move in the moving direction of the connector, and the retention lug is engaged in the retention hole in a state in which the own weight of the processing device is imposed on a lower portion of the retention hole.

A sixth aspect in combination with any of the above first to fifth aspects provides an ultrasonic diagnostic apparatus wherein a probe connection unit having a probe connector for connection of an ultrasonic wave transmitting and receiving probe is mounted removably to a probe connection unit mounting member arranged near the table.

A seventh aspect in combination with any of the above first to sixth aspects provides an ultrasonic diagnostic apparatus wherein a probe holder for holding the probe is mounted removably to a probe holder arranged near the table.

An eighth aspect in combination with any of the above first to seventh aspects provides an ultrasonic diagnostic apparatus further including a cable accommodating device for accommodating a cable of the probe.

A ninth aspect in combination with the above eighth aspect provides an ultrasonic diagnostic apparatus wherein the processing device and the probe connection unit are arranged above the cable accommodating device, and in the cable accommodating device, a portion where a height from the floor surface is 70 cm or less is 15 cm or less in thickness.

A tenth aspect in combination with the above eighth or ninth aspect provides an ultrasonic diagnostic apparatus wherein the probe connection unit is arranged above the cable accommodating device, the cable accommodating device has an aperture in a position 120 cm or more in height from the floor surface, and the cable of the probe is accommodated through the aperture into a cable accommodating space formed within the cable accommodating device.

An eleventh aspect in combination with any of the above first to tenth aspects provides an ultrasonic diagnostic apparatus wherein the operating device, the image display device and the processing device are mounted removably onto a movable carrier car.

A twelfth aspect in combination with any of the above first to eleventh aspects provides an ultrasonic diagnostic apparatus wherein the arm for mounting the image display device to the image display device mounting member or the arm for mounting the operating device to the operating device mounting member has a horizontal arm, the horizontal arm including a plurality of arm members connected rotatably in the horizontal direction, and a vertical arm capable of extension and retraction in the vertical direction.

A thirteenth aspect in combination with any of the above eighth to twelfth aspects provides an ultrasonic diagnostic apparatus wherein the cable accommodating device is provided with a cable stopper for preventing the entry and exit of the cable of the probe to and from the cable accommodating space.

A fourteenth aspect in combination with any of the above eighth to thirteenth aspects provides an ultrasonic diagnostic apparatus wherein a roller capable of abutment against a lower surface of the cable of the probe is provided in the aperture formed in the cable accommodating device.

A fifteenth aspect in combination with any of the above tenth to fourteenth aspects provides an ultrasonic diagnostic apparatus wherein the cable of the probe is bent in U shape from the aperture and is accommodated in the cable accommodating space.

A sixteenth aspect in combination with the above fifteenth aspect provides an ultrasonic diagnostic apparatus wherein a movable pulley is mounted on a lower end of the U shape of the cable accommodated in the cable accommodating space.

A seventeenth aspect in combination with any of the above first to sixteenth aspects provides an ultrasonic diagnostic apparatus wherein the image display device mounting member and the processing device mounting member are constituted by one and same mounting member.

An eighteenth aspect in combination with any of the above first to sixteenth aspects provides an ultrasonic diagnostic apparatus wherein the image display device mounting member and the operating device mounting member are constituted by one and same mounting member.

A nineteenth aspect in combination with any of the above first to sixteenth aspects provides an ultrasonic diagnostic apparatus wherein the operating device mounting member and the processing device mounting member are constituted by one and same mounting member.

A twentieth aspect in combination with any of the above first to sixteenth aspects provides an ultrasonic diagnostic apparatus wherein the image display device mounting member, the operating device mounting member and the processing device mounting member are constituted by one and same mounting member.

According to the invention of the above first aspect, the image display device is mounted through the arm to the interior of the table-installed room or to the image display device mounting member fixed within the installation room, the operating device is mounted to the operating device mounting member arranged near the table, and the processing device is mounted to the processing device mounting member arranged near the table, thus permitting the ultrasonic diagnostic apparatus to be used in a fixed state. Since the image display device, the operating device and the processing device are in a removable state, the ultrasonic diagnostic apparatus can be moved from the place where it is used in a fixed state to another place and can be used in the another place. Even in the case where the ultrasonic diagnostic apparatus is used in a fixed state, since the image display device is mounted through the arm having the movable portion, the image display device can be moved easily to for example a position at which an inspector can perform inspection with ease. Thus, the ultrasonic diagnostic apparatus employable fixedly at a predetermined place can be made more convenient for use.

The image display device, the operating device and the processing device are free in layout because they are in a removable state and they can be arranged to match the table-installed room and permit most effective utilization of space.

According to the invention of the above second aspect, the operating device is mounted through the arm to the interior of the table-installed room or to the operating device mounting member fixed within the table-installed room, the image display device is mounted to the image display device mounting member arranged near the table, and the processing device is mounted to the processing device mounting member arranged near the table, whereby the ultrasonic diagnostic apparatus can be used in a fixed state. Since the operating device, the image display device and the processing device are in a removable state, the ultrasonic diagnostic apparatus can be moved from the place where it is used in a fixed state to another place for use. Even when the ultrasonic diagnostic apparatus is used in a fixed state, since the operating member is mounted through the arm having the movable portion, the operating device can be moved easily for example to a position at which the inspector can perform inspection with ease. Thus, the ultrasonic diagnostic apparatus employable fixedly to a predetermined place can be made more convenient for use.

Moreover, the image display device, the operating device and the processing device are free in layout because they are in a removable state and can be arranged to match the table-installed room and permit most effective utilization of space.

According to the invention of the above third aspect, by adjusting the length of the columnar member to a length of abutment thereof in a pressed state against the ceiling and the floor surface of the installation room, the fixing of the columnar member to the installation room can be done in a simple manner.

According to the invention of the above fourth aspect, since at least one of the image display device and the processing device is arranged on the side opposite to the operating device which must be placed on the operator side with respect to the table, the operator-side space becomes wider than in the prior art because of absence of the image display device and the processing device, thus permitting effective utilization of space.

According to the invention of the above fifth aspect, after connecting the processing device to a connector provided in the mounting portion, the retention lug is retained on a lower portion of the retention hole in an imposed state of the own weight of the processing device, whereby the processing device is mounted to the processing device mounting member. In this case, since the connector is movable in a direction perpendicular to the floor surface, the processing device connected to the connector can be moved downward into engagement on the lower portion of the retention hole, whereby the mounting of the processing device can be done easily.

According to the invention of the above sixth aspect, when the ultrasonic diagnostic apparatus is used in a fixed state, the probe connection unit is mounted to the probe connection unit mounting member. On the other hand, since the probe connection unit is removable, the ultrasonic diagnostic apparatus can be moved from the place where it is used in a fixed state to another place for use.

According to the invention of the above seventh aspect, when the ultrasonic diagnostic apparatus is used in a fixed state, the probe holder is mounted to the probe hold mounting member. On the other hand, since the probe holder is removable, the ultrasonic diagnostic apparatus can be moved from the place where it is used in a fixed state to another place for use.

According to the invention of the above eighth aspect, since the cable of the probe can be accommodated in the probe accommodating device, it is possible to prevent the cable from being an obstacle.

According to the invention of the above ninth aspect, when the table is a bed for example, a portion of the cable accommodating device, whose height is higher than an average bed and in which a height thereof from the floor surface is 70 cm or less, is 15 cm or less in thickness, and the processing device and the probe connection unit are arranged above such a cable accommodating device. Therefore, if the gap between the table and the wall is set at, for example, 16 cm, the processing device and the probe connection unit can be arranged along the wall. Consequently, the space on the side opposite to the wall with respect to the table can be widened and it is possible to attain an effective utilization of space.

According to the invention of the above tenth aspect, an aperture of the cable accommodating device is formed at a position 120 cm or more in height from the floor surface, so when the table is for example a bed and if the height of an average bed and the body thickness of a subject are taken into account, it follows that the ingress and egress of the probe cable are performed from a position sufficiently higher than the subject laid on the bed. Thus, it is possible to prevent the cable from contacting the subject and giving an unpleasant feeling to the subject.

According to the invention of the above eleventh aspect, the operating device, the image display device and the processing device can be mounted onto a movable carrier car and can be moved thereby.

According to the invention of the above twelfth aspect, the horizontal arm includes a plurality of arm members connected rotatably in the horizontal direction and the vertical arm can extend and retract in the vertical direction and has a movable portion. Therefore, the image display device and the operating device can be moved easily to a position at which the inspector can conduct inspection with ease.

According to the invention of the above thirteenth aspect it is possible to prevent the cable from going out from the aperture or being pulled into the cable accommodating space against the operator's will.

According to the invention of the above fourteenth aspect, the ingress and egress of the cable to and from the aperture are smoothed by the roller.

According to the invention of the above fifteenth aspect, since the cable bent portion at a small curvature is only one portion that is the lower end of the U shape, it is possible to prevent damage of the cable.

According to the invention of the above sixteenth aspect it is possible to prevent the cable from being bent at a curvature smaller than the radius of the movable pulley.

According to the invention of the above seventeenth aspect, the image display device and the processing device are mounted to one and same mounting member.

According to the invention of the above eighteenth aspect, the image display device and the operating device are mounted to one and same mounting member.

According to the invention of the nineteenth aspect, the operating device and the processing device are mounted to one and same mounting member.

According to the invention of the twentieth aspect, the image display device, the operating device and the processing device are mounted to one and same mounting member.

Further objects and advantages of the embodiments described herein will be apparent from the following description as illustrated in the accompanying drawings.

### BRIEF DESCRIPTION OF THE DRAWINGS

FIG. 1 is a front view showing an ultrasonic diagnostic apparatus according to a first embodiment.

FIG. 2 is a side view of the ultrasonic diagnostic apparatus shown in FIG. 1.

FIG. 3 is a plan view of the ultrasonic diagnostic apparatus shown in FIG. 1.

FIG. 4 is a partially enlarged sectional view showing a mounting structure of an operating device in the ultrasonic diagnostic apparatus shown in FIG. 1.

FIG. 5 is a partially enlarged front view of a columnar member in the ultrasonic diagnostic apparatus shown in FIG. 1.

FIG. 6 is a sectional view taken on line A-A in FIG. 5.

FIG. 7 is a sectional view taken on line B-B in FIG. 5.

FIG. 8 is a rear view of a processing device in the ultrasonic diagnostic apparatus shown in FIG. 1.

FIG. 9 is a sectional view taken on line C-C in FIG. 8.

FIG. 10 is a partially enlarged plan view of a mounting member and a hold plate prior to mounting to a holder mounting arm.

FIG. 11 is a partially cut-away side view of the mounting member as attached to the hold mounting arm.

FIG. 12 is a side view showing the ultrasonic diagnostic apparatus to be moved for use.

FIG. 13 is a front view of the ultrasonic diagnostic apparatus shown in FIG. 12.

FIG. 14 is a front view showing an ultrasonic diagnostic apparatus according to a second embodiment.

FIG. 15 is a front view showing an ultrasonic diagnostic apparatus according to a third embodiment.

FIG. 16 is a side view of the ultrasonic diagnostic apparatus shown in FIG. 15.

FIG. 17 is a front view showing an ultrasonic diagnostic apparatus according to a fourth embodiment.

FIG. 18 is a side view of the ultrasonic diagnostic apparatus shown in FIG. 17.

FIG. 19 is a plan view of the ultrasonic diagnostic apparatus shown in FIG. 17.

FIG. 20 is a front view showing an ultrasonic diagnostic apparatus according to a fifth embodiment.

FIG. 21 is a partially cut-away side view of the ultrasonic diagnostic apparatus shown in FIG. 1.

FIG. 22 is a plan view of the ultrasonic diagnostic apparatus shown in FIG. 20.

FIG. 23 is a partially cut-away, enlarged side view showing an aperture and the vicinity thereof of the ultrasonic diagnostic apparatus shown in FIG. 20.

FIG. 24 is an enlarged front view showing apertures and the vicinity thereof of the ultrasonic diagnostic apparatus shown in FIG. 20.

FIG. 25 is an enlarged side view of a movable pulley and the vicinity thereof with a cover slid downward to expose the interior in the ultrasonic diagnostic apparatus shown in FIG. 20.

FIG. 26 is an enlarged side view of a movable pulley lock and the vicinity thereof in a released state of the movable pulley lock.

FIG. 27 is an enlarged side view of the movable pulley lock and the vicinity thereof in an operated state of the movable pulley lock.

FIG. 28 is an enlarged side view of the movable pulley and the vicinity thereof in a released state of a cable lock.

### DETAILED DESCRIPTION OF THE INVENTION

### First Embodiment

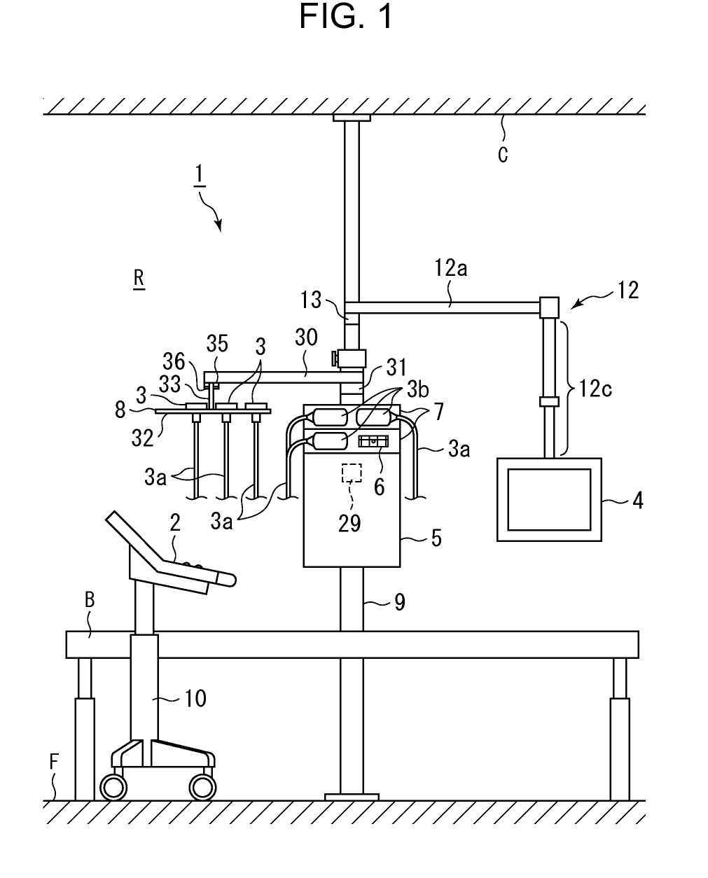

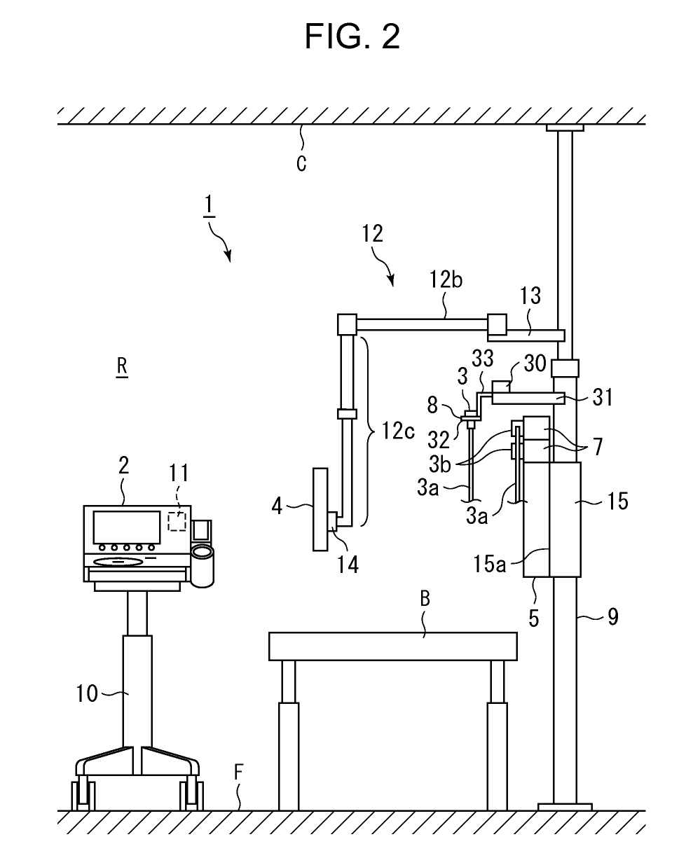

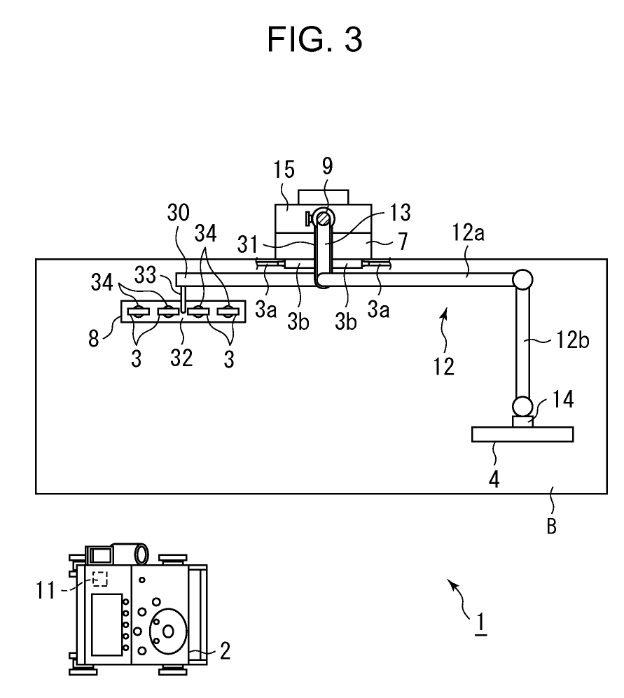

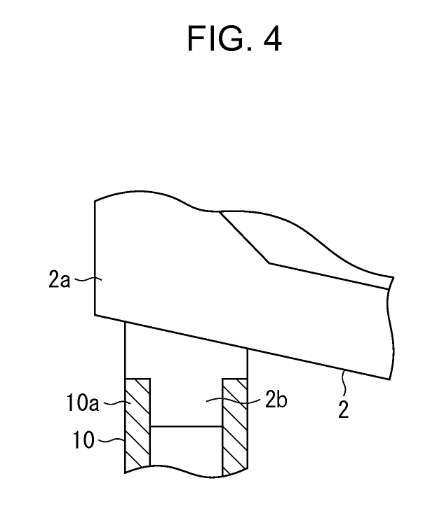

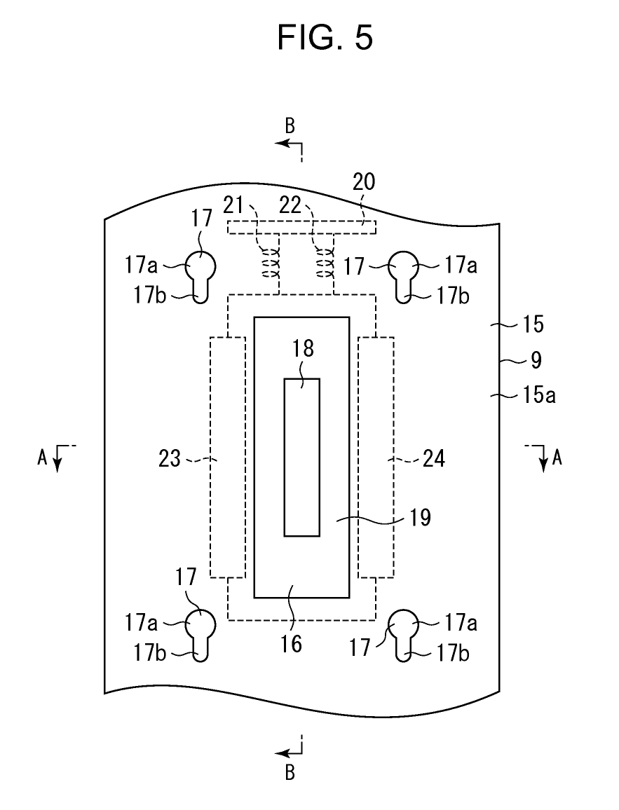

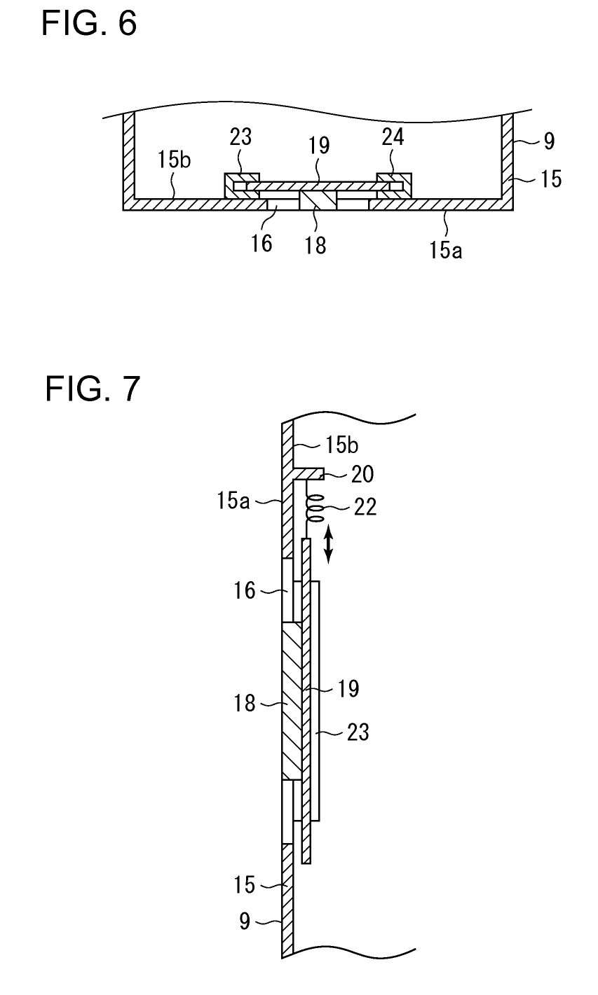

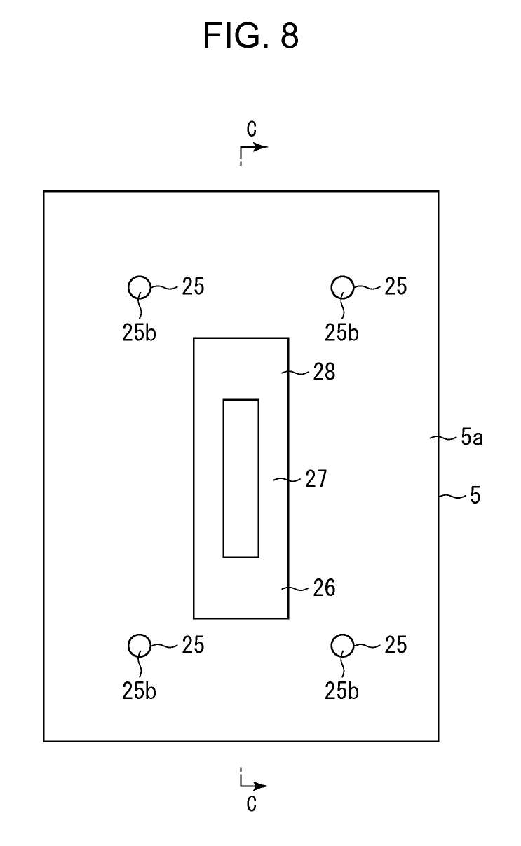

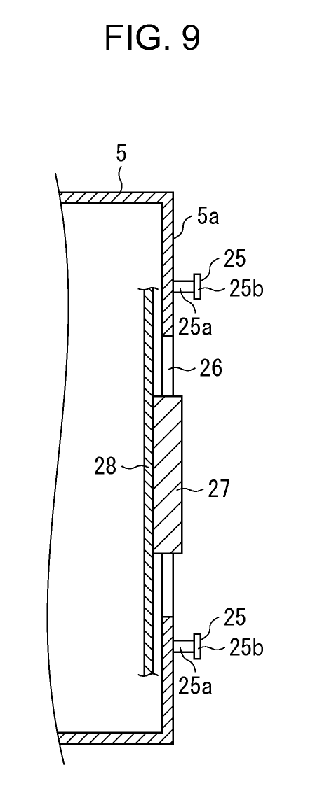

First, a first embodiment will be described below. FIG. 1 is a front view showing an ultrasonic diagnostic apparatus according to a first embodiment, FIG. 2 is a side view of the ultrasonic diagnostic apparatus shown in FIG. 1, FIG. 3 is a plan view of the ultrasonic diagnostic apparatus shown in FIG. 1, FIG. 4 is a partially enlarged sectional view showing an operating device mounting structure in the ultrasonic diagnostic apparatus shown in FIG. 1, FIG. 5 is a partially enlarged front view of a columnar member in the ultrasonic diagnostic apparatus shown in FIG. 1, FIG. 6 is a sectional view taken on line A-A in FIG. 5, FIG. 7 is a sectional view taken on line B-B in FIG. 5, FIG. 8 is a rear view of a processing device in the ultrasonic diagnostic apparatus shown in FIG. 1, and FIG. 9 is a sectional view taken on line C-C in FIG. 8.

An ultrasonic diagnostic apparatus 1 includes an operating device 2 for an operator to input an instruction, a probe 3 for transmission and reception of an ultrasonic wave, an image display device 4 for displaying an ultrasonic image, and a processing device 5 for making control to generate an ultrasonic image on the basis of an echo signal resulting from operation of the probe 3 in accordance with the operator's instruction and display the ultrasonic image on the image display device 4. The operating device 2, the image display device 4 and the processing device 5 are constituted as separate units.

The ultrasonic diagnostic apparatus 1 further includes a probe connection unit 7 having a probe connector 6 for connection thereto of the probe 3 and a probe holder 8 for holding the probe 3.

The ultrasonic diagnostic apparatus 1 not only can be used in a fixed state but also can be moved and used. In case of using it in a fixed state, as shown in FIGS. 1 to 3, the ultrasonic diagnostic apparatus 1 is provided with a columnar member 9 to which the image display device 4, processing device 5 and probe holder 8 are mounted.

The operating device 2 is attached removably to a movable base 10 with caster that is arranged near a table B (bed) for resting a subject thereon. The movable base 10 is an example of the operating device mounting member in the invention. The mounting structure of the operating device 2 will now be described with reference to FIG. 4. The operating device 2 has a body portion 2a and a projecting portion 2b projecting from a lower surface of the body portion 2a. The operating device 2 is mounted to the movable base 10 by fitting its projecting portion 2b into a tip end of a pipe-like portion 10a of the movable base 10.

The operating device 2 includes an operating device-side radio communication portion 11 for radio communication with the processing device 5. When the operator inputs an instruction through an operating device 2, the inputted instruction signal is transmitted to the processing device 5 by the operating device-side radio communication portion 11.

In this embodiment, the columnar member 9 is arranged on the side opposite to the arranged side of the operating device 2 with respect to the table B and near the table B. The columnar member 9 is capable of extension and retraction and, by being adjusted to a length of abutment thereof in a pressed state against both ceiling C and floor surface F of an installation room R with the table B installed therein, the columnar member 9 is fixed within the installation room R. The columnar member 9 is an example of the image display device mounting member, the processing device mounting member and the probe holder mounting member in the invention. That is, the image display device mounting member, the processing device mounting member and the probe holder mounting member are constituted by one and same mounting member.

The image display device 4 is mounted to the columnar member 9 through an arm 12. The arm 12 is attached to an arm mounting rod 13 provided in the columnar member 9. The arm 12 is dividedly made up of three arm members which are a first horizontal arm 12a, a second horizontal arm 12b and a vertical arm 12c.

The first horizontal arm 12a extends horizontally from the arm mounting rod 13. The second horizontal arm 12b extends horizontally from the first horizontal arm 12a. Further, the vertical arm 12c extends vertically from the second horizontal arm 12b.

The arm 12 will be further described. The first horizontal arm 12a is adapted to rotate horizontally in the connection thereof with the arm mounting rod 13. The first horizontal arm 12a and the second horizontal arm 12b are adapted to rotate horizontally in the respective connections. The vertical arm 12c is adapted to rotate horizontally in the connection thereof with the second horizontal arm 12b and is capable of extension and retraction. Since the arm 12 has these movable portions, it is possible to change the position of the image display device 6.

An image display device connector 14 is provided at a lower end of the vertical arm 12c and the image display device 4 is attached removably to the image display device connector 14.

The processing device 5 is mounted removably to a box-like portion 15 of the columnar member 9. The box-like portion 15 has a processing device mounting surface 15a that is perpendicular to the floor surface F and the processing device 5 is mounted to the processing device mounting surface 15a. The box-like portion 15 is an example of the mounting portion in the invention.

A mounting structure of the processing device 5 will now be described with reference to FIGS. 5 to 9. The details of an internal configuration are omitted in the sectional views of FIGS. 6, 7 and 9. A mounting member-side window 16 is formed in the processing device mounting surface 15a and four retention holes 17 are formed around the mounting member-side window 16. The retention holes 17 are each substantially in the shape of a key hole and are each provided with a circular hole portion 17a and an elongated hole portion 17b. The elongated hole portion 17b is formed in a direction perpendicular to the floor surface F which direction is a moving direction of a mounting member-side connector 18 provided on a board 19 to be described later.

The mounting member-side connector 18 with the processing device 5 connected thereto is arranged in the mounting member-side window 16 so as to be movable in a direction (vertical direction) perpendicular to the floor surface. More specifically, the mounting member-side connector 18 is provided on the board 19. The board 19 is arranged at a position at which a processing device-side connector 27 to be described later of the processing device 5 can be connected to the mounting member-side connector 18 when disc portions 25b of retention lugs 25 to be described later which are formed on a rear surface 5a of the processing device 5 are inserted into the box-like portion 15 through circular hole portions 17a. The board 19 is secured to springs 21 and 22 that are fixed to a projecting portion 20 projecting from an inner wall surface 15b of the box-like portion 15. The board 19 is movable in the direction perpendicular to the floor surface.

The movement of the board 19 in the direction perpendicular to the floor surface is guided by guide members 23 and 24 of a turned square U-shaped section mounted on the inner wall surface 15b. Also, the movement of the board 19 in a pressing direction against the mounting member-side connector 18 is inhibited by the guide members 23 and 24 when connecting the processing device-side connector 27 to the mounting member-side connector 18.

On the other hand, retention lugs 25 are formed on the rear surface 5a of the processing device 5. The retention lugs 25 each include a rod portion 25a and a disc portion 25b provided at the tip of the rod portion 25a. The rod portion 25a is smaller in diameter than at least each of the elongated hole portion 17b. The disc portion 25b has a diameter larger than each of the elongated hole portion 17b but smaller than each of the circular hole portions 17a.

A processing device-side window 26 is formed in the rear surface of the processing device 5 and the processing device-side connector 27 is arranged in the processing device-side window 26. The processing device-side connector 27 is provided on a board 28 that is fixed within the processing device 5.

A description will now be given about mounting the processing device 5 to the box-like portion 15. First, the disc portions 25b of the retention lugs 25 in the processing device 5 are inserted into the box-like portion 15 through the circular hole portions 17a of the retention holes 17 in the box-like portion 15. At this time, the processing device-side connector 27 is connected to the mounting member-side connector 18. Then, with the processing device-side connector 27 connected to the mounting member-side connector 18, the processing device 5 is moved down against the biasing force of the springs 21 and 22. At this time, the rod portions 25a of the retention lugs 25 moves through the elongated hole portions 17b. Then, the retention lugs 25 are brought into engagement in lower portions of the elongated hole portions 17b respectively in an imposed state of the own weight of the processing device 5. By thus adopting the mounting structure permitting movement of the mounting member-side connector 18 it becomes easier to effect connection between the mounting member-side connector 18 and the processing device-side connector 27, thereby permitting easy mounting of the processing device 5.

Turning back to FIG. 1, the processing device 5 has a processing device-side radio communication portion 29 for radio communication with the operating device 2. Data on an ultrasonic image generated in the processing device 5 are inputted to the image display device 4 through a cable (not shown) laid within the columnar member 9 and arm 12.

In this embodiment, two probe connection units 7 are stacked one on the other and are in this state mounted on the processing device 5 removably. The processing device 5 is an example of the probe connection unit mounting member in the invention. The processing device 5 and the probe connection units 7 are electrically connected with each other.

A cable connector 3b provided at an end of a probe cable 3a of the probe 3 is connected to the probe connector 6 of each probe connection unit 7. In this embodiment, each probe connection unit 7 is provided with two probe connectors 6 and therefore a maximum of four probes 3 can be connected.

The probe holder 8 is attached to the columnar member 9 through a holder mounting arm 30. The holder mounting arm 30 is attached to a holder mounting rod 31 which is provided in the columnar member 9.

The holder mounting arm 30 extends horizontally from the holder mounting rod 31 and is rotatable horizontally in the mounting portion of the hold mounting rod 31.

The probe holder 8 has a hold plate 32, which plate is attached to the holder mounting arm 30 removably by a mounting member 33. The hold plate 32 is formed with four hold holes 34. Grip portions of the probes 3 are inserted into the hold holes 34 respectively and tip ends of the probes are engaged with an upper surface of the hold plate 32. In this state the probes 3 are held by the hold plate 32.

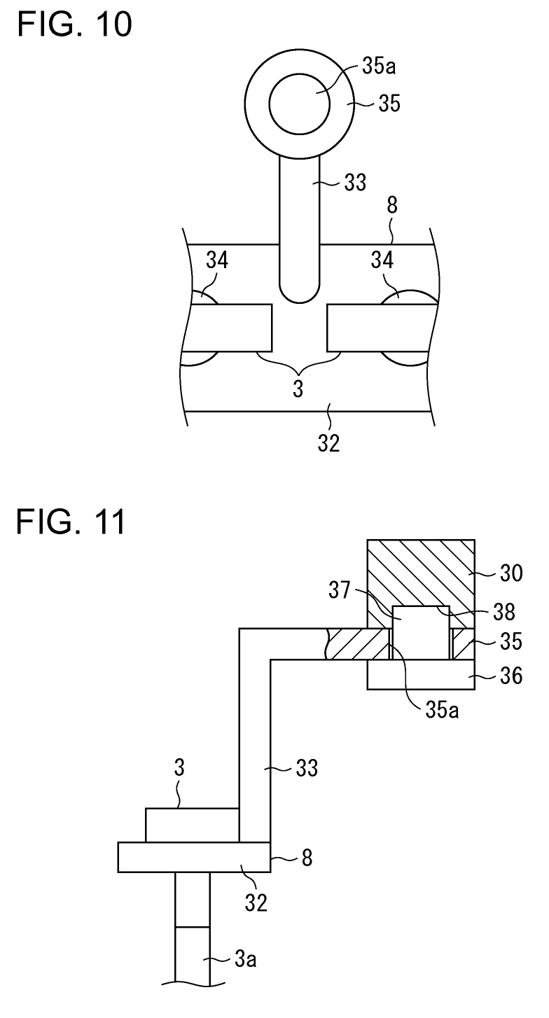

Now, with reference to FIGS. 10 and 11, a description will be given about a mounting structure for mounting the hold plate 32 to the holder mounting arm 30 with use of the mounting member 33. FIG. 10 is a partially enlarged plan view of both mounting member 33 and hold plate 32 prior to mounting to the holder mounting arm 30 and FIG. 11 is a partially cut-away side view in a mounted state of the mounting member 33 to the holder mounting arm 30.

The mounting member 33 is L-shaped and on one end side thereof it is integral with the hold plate 32. The mounting member 33 is provided on the opposite end side with a ring portion 35. With a cap member 36, the ring portion 35 is secured to the holder mounting arm 30. More particularly, a projecting portion 37 of the cap member 36 is inserted into a hollow portion 35a of the ring portion 35 and in this state it is fitted in a fitting hole 38 formed in the holder mounting hole 30, whereby the mounting member 33 is mounted to the holder mounting arm 30.

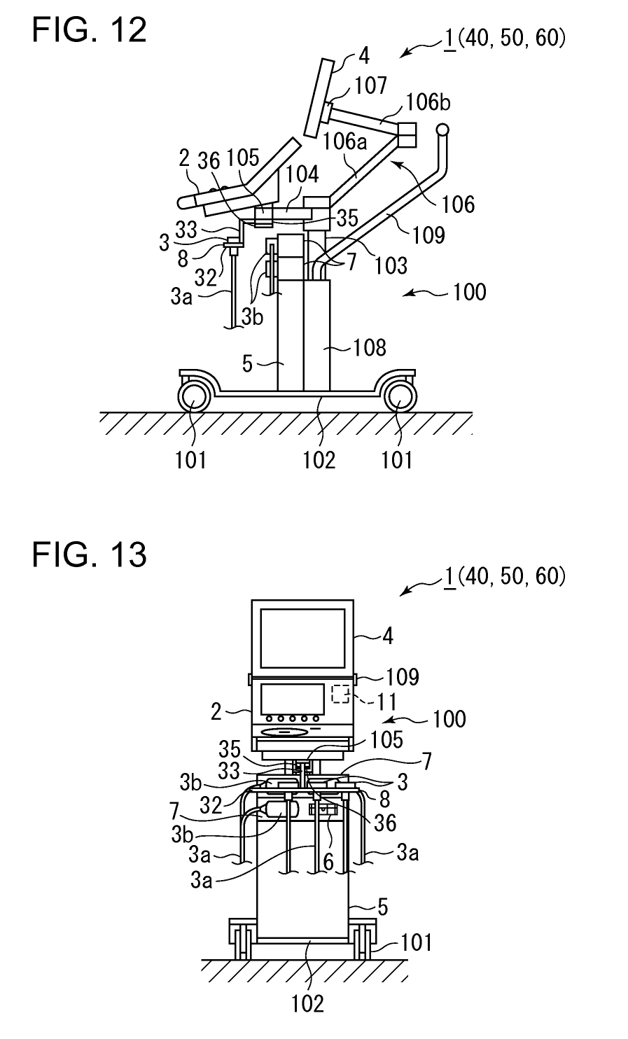

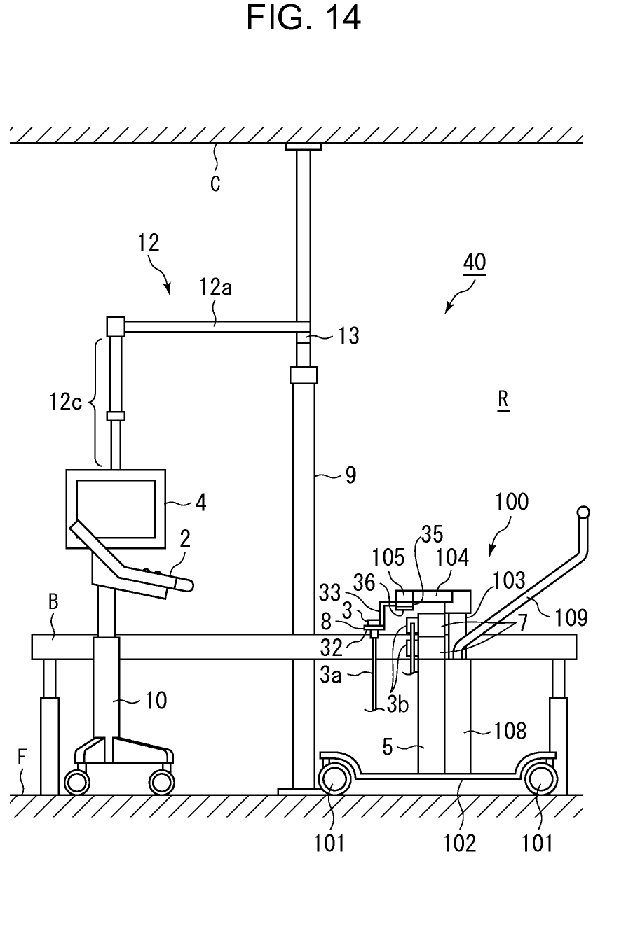

A description has been given above about the case where the ultrasonic diagnostic apparatus 1 is used in a fixed state. Next, with reference to FIGS. 12 and 13, a description will be given about the case where the ultrasonic diagnostic apparatus 1 is moved and used. FIG. 12 is a side view showing the ultrasonic diagnostic apparatus 1 to be moved and used and FIG. 13 is a front view of the ultrasonic diagnostic apparatus shown in FIG. 12.

In the ultrasonic diagnostic apparatus 1, the operating device 2, the image display device 4, the processing device 5, the probe connection units 7 and the probe holder 8 are mounted removably onto a movable carrier car 100, whereby the ultrasonic diagnostic apparatus 1 can be moved and used. A description will now be given about the configuration of the movable carrier car 100. In this movable carrier car, a support rod 103 is provided on a base portion 102 that has four casters 101.

The operating device 2 is attached removably to a mounting rod 104 that is provided on the support rod 103. A tip end of the mounting rod 104 is formed as a ring portion 105 and, like being mounted to the pipe-like portion 10a of the movable base 10, the projecting portion 2b(not shown in FIGS. 12 and 13) of the operating device 2 is fitted in the ring portion 105 removably, whereby the operating device 2 is mounted.

The probe holder 8 is mounted to the mounting rod 104 by the same mounting structure as that adopted for mounting to the holder mounting arm 30. That is, the projecting portion 37 (not shown in FIGS. 12 and 13) of the cap member 36 is inserted into the hollow portion 35a of the ring portion 35 of the mounting member 33 and in this state it is fitted in the ring portion 105 of the mounting rod 104, whereby the mounting member 33 is secured to the mounting rod 104.

The image display device 4 is attached to the support rod 103 through an arm member 106. The arm member 106 includes a first member 106a and a second member 106b and is generally dog-leg shaped. The first member 106a is attached rotatably to an upper end of the support rod 103. The first and second members 106a, 106b are connected together rotatably. At a tip end of the second member 106b there is provided a connector 107 to which the image display device 4 is attached removably.

At a lower portion of the support rod 103 there is provided a box-like portion 108 of the same configuration as the box-like portion 15 of the columnar member 9. Like the mounting structure in the box-like portion 15 of the columnar member 9, the processing device 5 is attached to the box-like portion 108. Illustration of its mounting structure in the drawing is omitted. Ultrasonic image data generated in the processing device 5 are inputted to the image display device 4 through a cable (not shown) that is laid in both support rod 103 and arm member 106.

A grip member 109 is attached to the box-like portion 108 and the movable carrier car 100 can be moved with the grip member 109.

The probe connection unit 7 is mounted removably onto the processing device 5 in the same manner as in the fixed state.

According to the first embodiment described above, the image display device 4, the processing device 5 and the probe holder 8 are attached to the columnar member 9 which is fixed near the table B within the installation room R, the probe connection unit 7 is attached to the processing device 5, and the operating device 2 is attached to the movable base 10 arranged near the table B, whereby the ultrasonic diagnostic apparatus 1 can be used in its fixed state. On the other hand, by removing the image display device 4 from the arm 12, removing the processing device 5 from the box-like portion 15, removing the probe holder 8 from the holder mounting arm 30 and removing the operating device 2 from the movable base 10 and mounting them to the movable carrier car 100, the ultrasonic diagnostic apparatus 1 can be moved from the place where it is used in its fixed state to another place for use. Even when the ultrasonic diagnostic apparatus is used in its fixed state, since the image display device 4 is mounted through the arm 12 that is rotatable horizontally and capable of extension and retraction vertically, the image display device 4 can be moved easily to for example a position at which the inspector can perform inspection with ease. Thus, the ultrasonic diagnostic apparatus 1 which is employable in a fixed state to a predetermined place can be made more convenient for use.

By adjusting the length of the columnar member 9 to a length of abutment in a pressed state against both ceiling C and floor surface F of the installation room R, the columnar member 9 can be fixed easily to the installation room R. Besides, since the image display device 4 and the processing device 5 are arranged on the side opposite to the operating device 2 which must be placed on the operator side with respect to the table B, the operator-side space becomes wider than in the prior art as a result of absence of both image display device 4 and processing device 5 and thus it is possible to effectively utilize the space of the installation room R.

### Second Embodiment

A second embodiment will now be described with reference to FIG. 14. FIG. 14 is a front view showing an ultrasonic diagnostic apparatus according to a second embodiment. In FIG. 14, the same constructional portions as in the first embodiment are identified by the same reference numerals as in the first embodiment, and detailed explanations thereof will here be omitted.

When the ultrasonic diagnostic apparatus, indicated at 40, of this second embodiment is used in a fixed state, the image display device 4 is attached to the columnar member 9, but the processing device 5 and the probe holder 8 are mounted to the movable carrier car 100. That is, the movable carrier car 100 is an example of the processing device mounting member and the probe holder mounting member. In this second embodiment, the movable carrier car 100 is arranged near the table B and on the side where the operating device 2 is placed with respect to the table B.

In this second embodiment, radio communication is performed between the processing device 5 and the image display device 4, and ultrasonic image data generated in the processing device 5 are transmitted by radio to the image display device 4.

When the ultrasonic diagnostic apparatus 40 is to be moved for use, the operating device 2, the image display device 4, the processing device 5, the probe connection unit 7 and the probe holder 8 are attached to the movable carrier car 100 as in the first embodiment (see FIGS. 12 and 13).

According to the ultrasonic diagnostic apparatus 40 of this second embodiment described above, the image display device 4 is attached to the columnar member 9, the operating device 2 is attached to the movable base 10 which is arranged near the table B, and the processing device 5, the probe connection unit 7 and the probe holder 8 are attached to the movable carrier car 100 which is arranged near the table B, whereby the ultrasonic diagnostic apparatus 40 can be used in its fixed state. On the other hand, by removing the image display device 4 from the arm 12 and removing the operating device 2 from the movable base 10 and attaching them to the movable carrier car 100, the ultrasonic diagnostic apparatus 40 can be moved from the place where it is used in its fixed state to another place for use.

Moreover, since the image display device 4 is arranged on the side opposite to the operating device 2 which must be arranged on the operator side with respect to the table B, the space on the operator side becomes wider than in the prior art as a result of absence of the image display device 4 and thus it is possible to effectively utilize the space of the installation room R. Further, there can be obtained the same other effects as in the first embodiment.

Third Embodiment

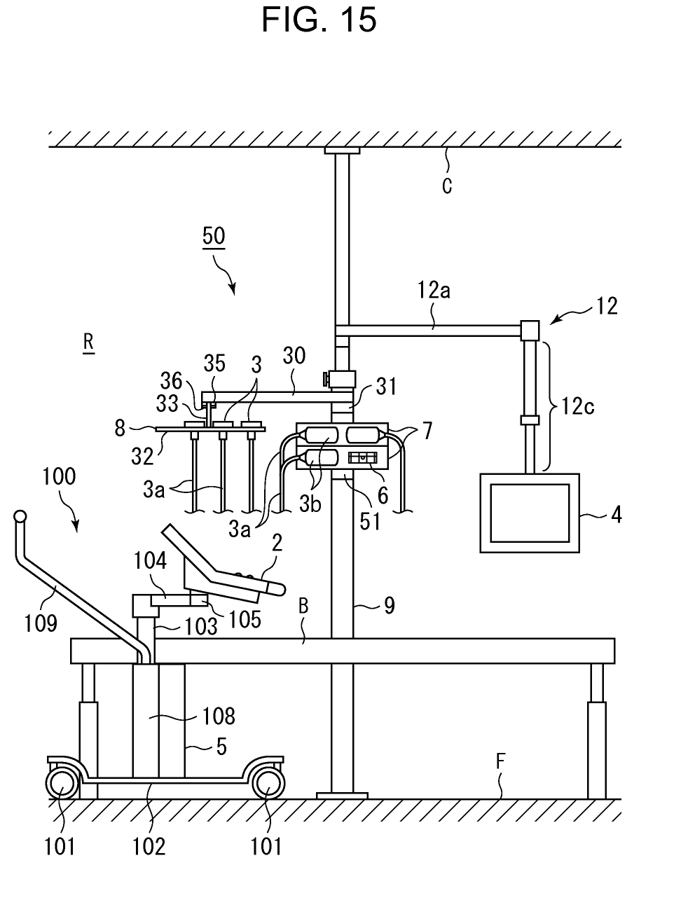

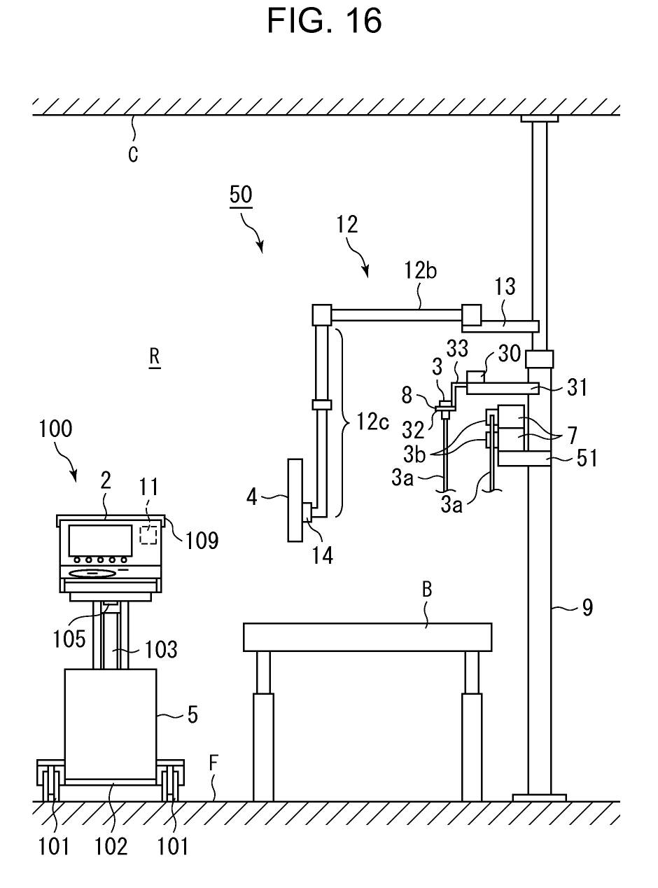

A third embodiment will now be described with reference to FIGS. 15 and 16. FIG. 15 is a front view showing an ultrasonic diagnostic apparatus according to a third embodiment and FIG. 16 is a side view of the ultrasonic diagnostic apparatus shown in FIG. 15. In FIGS. 15 and 16, the same constructional portions as in the first and second embodiments are identified by the same reference numerals as in those embodiments, and detailed explanations thereof will here be omitted.

In the ultrasonic diagnostic apparatus, indicated at 50, of this third embodiment, the probe connection unit 7 is attached removably to a connection unit mounting rod 51 provided in the columnar member 9. That is, the probe connection unit 7 is attached to the columnar member 9 through the connection unit mounting rod 51. In this third embodiment, the columnar member 9 serves as a probe connection unit mounting member.

As in the first embodiment, the image display device 4 and the probe holder 8 are attached to the columnar member 9. That is, the image display device mounting member, the probe holder mounting member and the probe connection unit mounting member are constituted by one and same mounting member.

When the ultrasonic diagnostic apparatus 50 is to be used in its fixed state, the operating device 2 and the processing device 5 are attached to the movable carrier car 100. That is, the movable carrier car 100 is an example of the operating device mounting member and the processing device mounting member. The movable carrier car 100 is arranged near the table B and on the side opposite to the columnar member 9 with respect to the table B.

In this third embodiment, the probe connection unit 7 performs radio communication with the processing unit and a signal resulting from transmission or reception of an ultrasonic wave is sent by radio to the processing device 5. Ultrasonic image data generated in the processing device 5 are sent to the image display device 4.

The probe connection unit 7 may be supplied with electric power from a power supply (not shown) arranged within the installation room R. The probe connection unit 7 may have a battery power supply.

When the ultrasonic diagnostic apparatus 50 of this third embodiment is to be moved for use, the operating device 2, the image display device 4, the processing device 5, the probe connection unit 7 and the probe holder 8 are attached to the movable carrier car 100 as in the first and second embodiments (see FIGS. 12 and 13).

According to the ultrasonic diagnostic apparatus 50 of this third embodiment described above, the image display device 4, the probe connection unit 7 and the probe holder 8 are attached to the columnar member 9, while the operating member 2 and the processing device 5 are attached to the movable carrier car 100 which is arranged near the table B, whereby the ultrasonic diagnostic apparatus 50 can be used in its fixed state. On the other hand, by removing the image display device 4 from the arm 12, removing the probe connection unit 7 from the connection unit mounting rod 51, removing the probe holder 8 from the holder mounting arm 30 and attaching them to the movable carrier car 100, the ultrasonic diagnostic apparatus 50 can be moved from the place where it is used in its fixed state to another place for use.

Moreover, since the image display device 4 is arranged on the side opposite to the operating device 2 which must be placed on the operator side with respect to the table B, the operator-side space becomes wider than in the prior art as result of absence of the image display device 4 and it is possible to effectively utilize the space of the installation room R. Further, it is possible to obtain the same effects as in the first and second embodiments.

### Fourth Embodiment

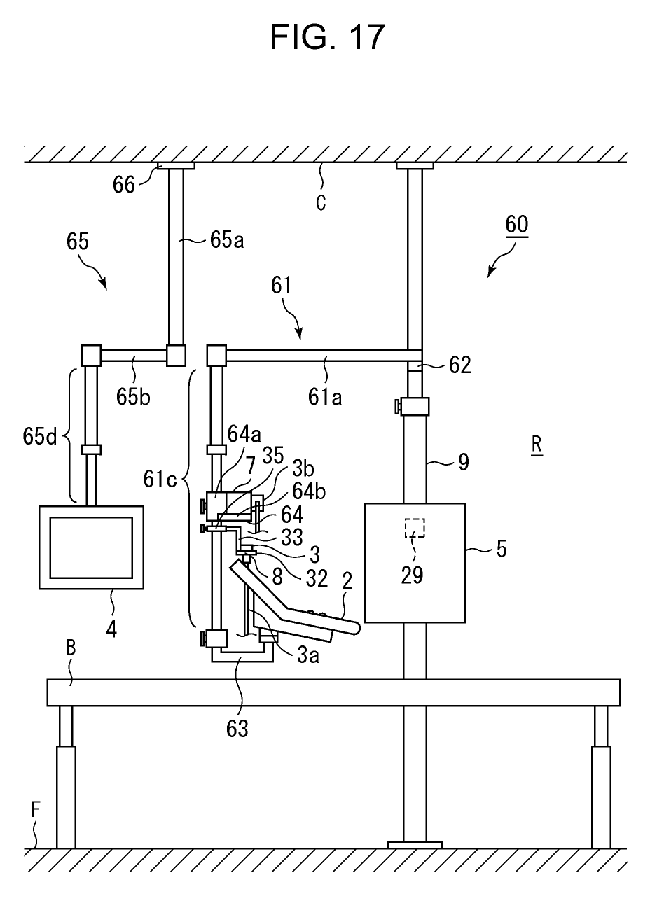

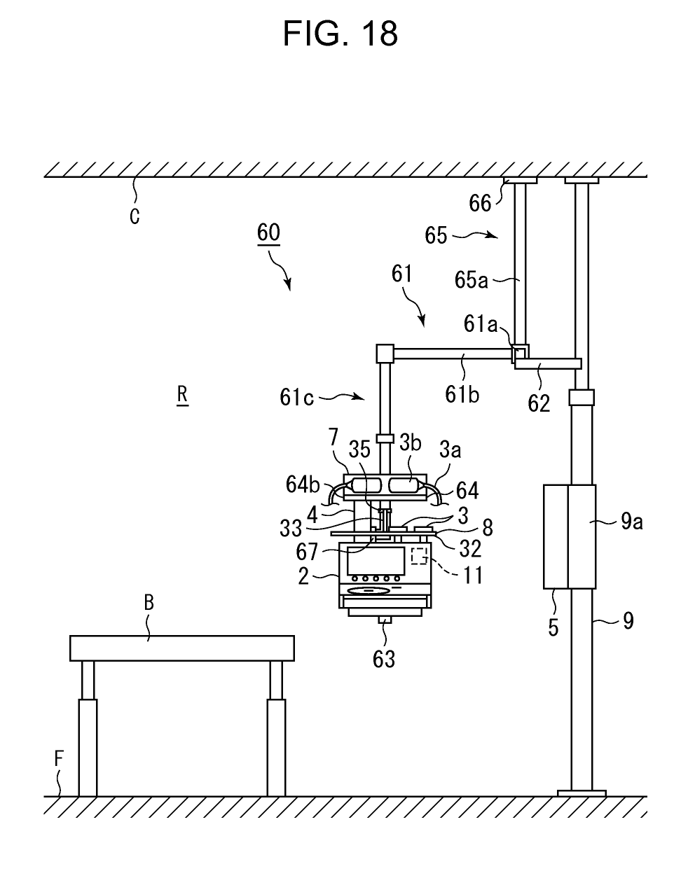

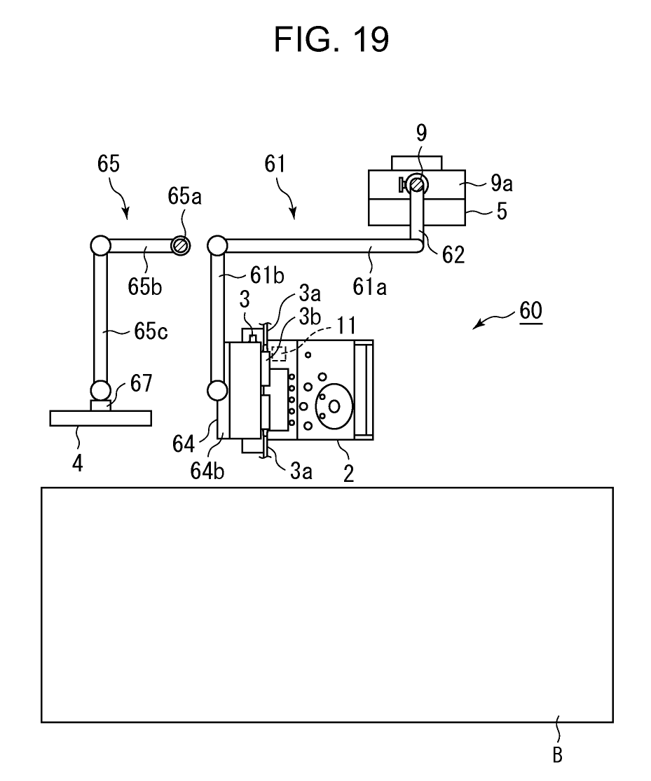

A fourth embodiment will now be described with reference to FIGS. 17 to 19. FIG. 17 is a front view showing an ultrasonic diagnostic apparatus according to a fourth embodiment, FIG. 18 is a side view of the ultrasonic diagnostic apparatus shown in FIG. 17, and FIG. 19 is a plan view of the ultrasonic diagnostic apparatus shown in FIG. 17. In FIGS. 17 to 19, the same constructional portions as in the first and second embodiments are identified by the same reference numerals as in those embodiments, and detailed explanations thereof will be omitted.

In the ultrasonic diagnostic apparatus, indicated at 60, of this fourth embodiment, the operating device 2, the probe connection unit 7 and the probe holder 8 are attached to the columnar member 9 through an arm 61. The columnar member 9 is an example of the operating device mounting member, the probe connection unit mounting member and the probe holder mounting member in the invention. That is, the operating device mounting member, the probe connection unit mounting member and the probe holder mounting member are constituted by one and same mounting member.

The arm 61 is attached to an arm mounting rod 62 provided in the columnar member 9. The arm 61 is dividedly made up of three arm members which are a first horizontal arm 61a, a second horizontal arm 61b and a vertical arm 61c.

The first horizontal arm 61a extends horizontally from the arm mounting rod 62. The second horizontal arm 61b extends horizontally from the first horizontal arm 61a. Further, the vertical arm 61c extends vertically from the second horizontal arm 61b.

A further description will now be given about the arm 61. The first horizontal arm 61 a is adapted to rotate horizontally in its connection with the arm mounting rod 62 and the first and second horizontal arms 61a, 61b are adapted to rotate horizontally in the respective connections. Further, the vertical arm 61c is adapted to rotate horizontally in its connection with the second horizontal arm 61b and is capable of extension and retraction. Since the arm 61 has these movable portions, the positions of the operating device 2, the probe connection unit 7 and the probe holder 8 can be changed.

A generally turned square U-shaped connecting member 63 is attached removal to a lower end of the vertical arm 61c. The operating device 2 is attached to the vertical arm 61 c through the connecting member 63.

The vertical arm 61c is removably fitted in one end of the connecting member 63 and the projecting portion 2b (not shown in FIGS. 17 to 19) of the operating device 2 is removably fitted in an opposite end of the connecting member 63.

The probe connection unit 7 and the probe holder 8 are also attached to the vertical arm 61c. The probe connection unit 7 is attached to the vertical arm 61 c through a connection unit mounting member 64. The connection unit mounting member 64 includes a ring portion 64a for fitting with the vertical arm 61 and a plate portion 64b provided in the ring portion 64a. The probe connection unit 7 is attached to the plate portion 64b removably.

As in the third embodiment, a signal resulting from transmission or reception of an ultrasonic wave is sent by radio to the processing device 5 from the probe connection unit 7.

By fitting the vertical arm 61c removably with the ring portion 35 of the mounting member 33 the probe holder 8 is mounted to the vertical arm 61c.

The processing device 5 is also attached to the columnar member 9. The columnar member 9 is an example of the processing device mounting member in the invention. Therefore, the processing device mounting member, the operating device mounting member, the probe connection unit mounting member and the probe holder mounting member are constituted by one and same mounting member. The processing device 5 is attached to a box portion 9a of the columnar member 9. Ultrasonic image data generated in the processing device 5 are sent by radio to the image display device 4.

The image display device 4 is arranged on the same side as the arranged side of the columnar member 9 with respect to the table B and is attached to the ceiling C of the installation room R through an arm 65. The arm 65 is dividedly made up of four arm members which are a first vertical arm 65a, a first horizontal arm 65b, a second horizontal arm 65c and a second vertical arm 65d. The image display device 4 is mounted removably to a lower end of the second vertical arm 65d.

The first vertical arm 65a is provided on a base member 66 fixed to the ceiling C and extends perpendicularly from the base member 66. The first horizontal arm 65b extends horizontally from the first vertical arm 65a. The second horizontal arm 65c extends horizontally from the first horizontal arm 65b. Further, the second vertical arm 65d extends perpendicularly from the second horizontal arm 65c.

A further description will now be given about the arm 65. The first horizontal arm 65b is adapted to rotate horizontally at its connection with the first vertical arm 65a and the first and second horizontal arms 65b, 65c are adapted to rotate horizontally in the respective connections. The second vertical arm 65d is adapted to rotate horizontally in its connection with the second horizontal arm 65c and is capable of extension and retraction.

An image display device connector 67 is provided at a lower end of the second vertical arm 65d and the image display device 4 is attached to the image display device connector 67 removably.

When the ultrasonic diagnostic apparatus 60 of this fourth embodiment is to be moved for use, the operating device 2, the image display device 4, the processing device 5, the probe connection unit 7 and the probe holder 8 are attached to the movable carrier car 100 as in the first to third embodiments (see FIGS. 12 and 13).

According to the ultrasonic diagnostic apparatus 60 of this fourth embodiment described above, the operating device 2, the processing device 5, the probe connection unit 7 and the probe holder 8 are attached to the columnar member 9, and the image display device 4 is attached to the ceiling C, whereby the ultrasonic diagnostic apparatus 60 can be used in its fixed state. On the other hand, by removing the operating device 2 from the connecting member 63, removing the processing device 5 from the box portion 9a, removing the probe connection unit 7 and the probe holder 8 from the vertical arm 61c, further removing the image display device 4 from the second vertical arm 65d and attaching them to the movable carrier car 100 like the first to third embodiments, the ultrasonic diagnostic apparatus 60 can be moved from the place where it is used in its fixed state to another place for use. Even when the ultrasonic diagnostic apparatus 60 is used in its fixed state, since the operating device 2 and the image display device 4 are mounted through the arms 61 and 65 which are rotatable in the horizontal direction and capable of extension and retraction in the vertical direction, the operating device 2 and the image display device 4 can be moved for example to a position at which the inspector can perform inspection easily. Thus, as in the first to third embodiments, the ultrasonic diagnostic apparatus 60 that is employable in a fixed state to a predetermined place can be made more convenient for use.

Further, as in the first to third embodiments, the columnar member 9 can be fixed to the installation room R in a simple manner.

### Fifth Embodiment

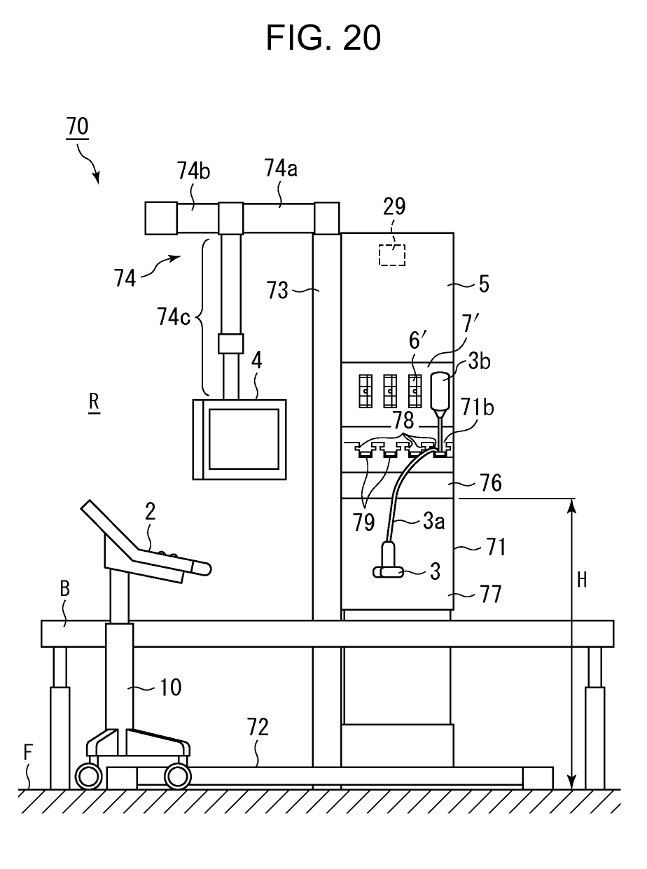

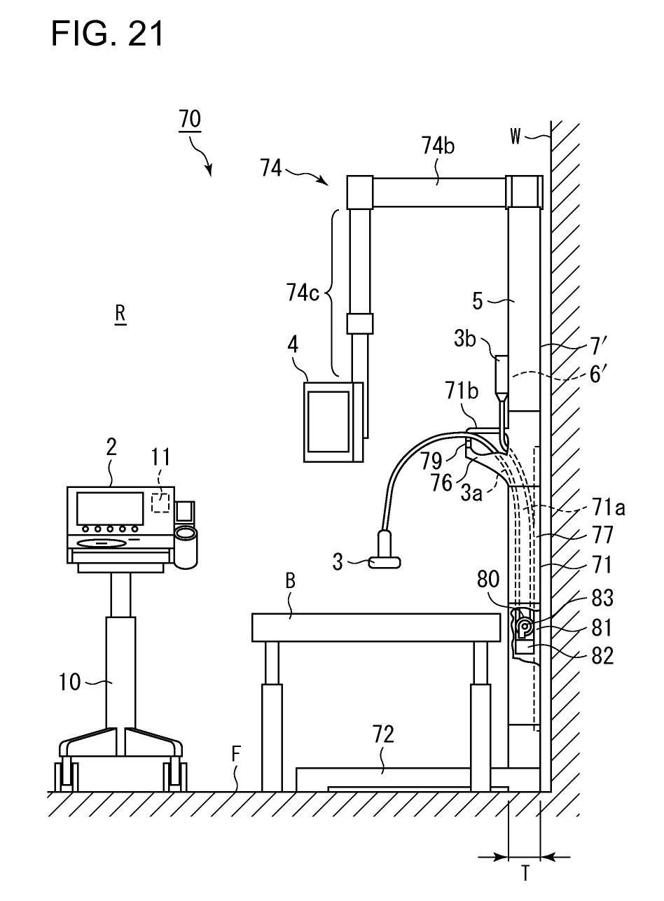

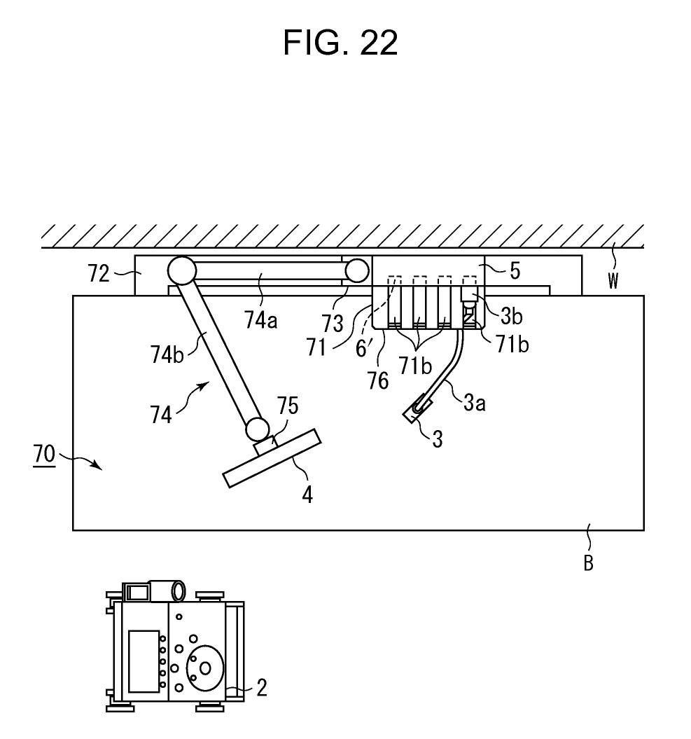

A fifth embodiment will now be described. FIG. 20 is a front view showing an ultrasonic diagnostic apparatus according to a fifth embodiment, FIG. 21 is a partially cut-away side view of the ultrasonic diagnostic apparatus shown in FIG. 1, and FIG. 22 is a plan view of the ultrasonic diagnostic apparatus shown in FIG. 20.

When the ultrasonic diagnostic apparatus, indicated at 70, of this fifth embodiment is to be used in a fixed state, the operating device 2, as in the first embodiment, is attached removably to the movable base 10 that is arranged near the table B.

On the side opposite to the arranged side of the operating device 2 with respect to the table B the ultrasonic diagnostic apparatus 70 of this embodiment is provided with a cable accommodating device 71 for accommodating the probe cable 3a of the probe 3. The cable accommodating device 71 is provided on an anti-fall leg 72 which is turned square U-shaped in plan. The anti-fall leg 72 may be fixed to the floor surface F with anchor bolts (not shown). As to the cable accommodating device 71, a detailed description will be given later.

A probe connection unit 7′ is mounted removably onto the cable accommodating device 71. That is, the cable accommodating device 71 is here an example of the probe connection unit mounting member in the invention. In this embodiment the probe connection unit 7′ has four probe connectors 6′. The direction of the probe connectors 6′ is different 90° from that of the probe connectors 6 provided in the probe connection unit 7 in each of the above embodiments.

The processing unit 5 is connected removably onto the probe connection unit 7′. That is, the probe connection unit 7′ is here an example of the processing device mounting member in the invention. The processing device 5 and the probe connection unit 7′ are electrically connected with each other through a cable (not shown) for example.

A support rod 73 is fixed to a side face of the cable accommodating device 71. The image display device 4 is attached to the support rod 73 through an arm 74. That is, the support rod 73 is here an example of the image display device mounting member in the invention.

The arm 74 includes three arm members which are a first horizontal arm 74a extending from the support rod 73, a second horizontal arm 74b extending horizontally from the first horizontal arm 74a and a vertical arm 74c extending vertically from the second horizontal arm 74b. The first horizontal arm 74a is adapted to rotate horizontally in its connection to the support rod 73. The first horizontal arm 74a and the second horizontal arm 74b are adapted to rotate horizontally in the respective connections. Further, the vertical arm 74c is adapted to rotate horizontally in its connection to the second horizontal arm 74b and is capable of extension and retraction.

An image display device connector 75 is provided at a lower end of the vertical arm 74c and the image display device 4 is attached removably to the image display device connector 75. Ultrasonic image data generated in the processing device 5 are inputted to the image display device 4 through a cable (not shown) that is laid within both support rod 73 and arm 74.

A description will now be given about the cable accommodating device 71. The cable accommodating device 71 is approximately 150.0 centimeters (cm) high from the floor surface F and its portion up to a height H of approximately 100.0 cm (higher than the sum of the height of the table B and the body thickness of an average subject) from the floor surface F is approximately 10.0 cm in thickness T, provided in the invention the thickness T of the portion of the cable accommodating device 71 up to the portion of the height H of approximately 100.0 cm from the floor surface F may be approximately 15.0 cm or less. The cable accommodating device 71 is formed as a projecting portion 76 in its range of between approximately 100.0 cm and approximately 130.0 cm in height from the floor surface F. The thickness of the projecting portion 76 is approximately 20.0 cm.

A vertically slidable cover 77 is attached to the cable accommodating device 71. By sliding the cover 77 the interior of the cable accommodating device 71 becomes exposed in its range between approximately 65.0 and approximately 100.0 cm in height from the floor surface F, so that the operator can make access to a cable accommodating space 71a formed within the cable accommodating device 71. As a result, it becomes easier to carry out the maintenance work and it becomes possible to remove and mount the probe cable 3a of the probe 3 from and to a movable pulley 80 to be described later.

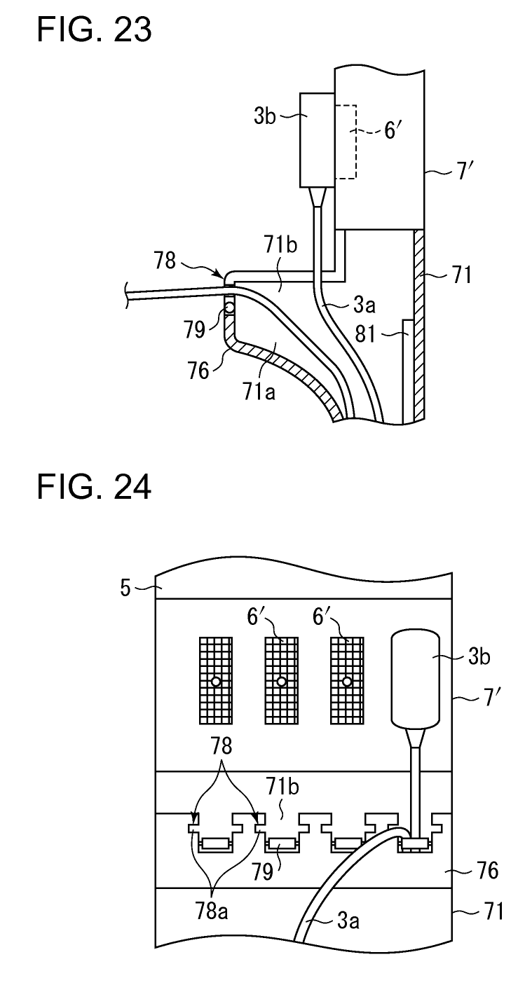

FIG. 23 is a partially cut-away enlarged side view showing an aperture and the vicinity thereof in the ultrasonic diagnostic apparatus 70 shown in FIG. 20 and FIG. 24 is an enlarged front view showing apertures and the vicinity thereof in the ultrasonic diagnostic apparatus 70 shown in FIG. 20. The projecting portion 76 of the cable accommodating device 71 is formed with apertures 71b in its range between approximately 120.0 cm and approximately 130.0 cm in height from the floor surface F. The cable connector 3b is connected to the probe connector 6′ which lie in a position higher than the apertures 71b and the probe cable 3a is accommodated into the cable accommodating space 71 a through the associated aperture 71b.

The probe cable 3a is accommodated in a U-bent state within the cable accommodating space 71a. Since the probe cable 3a is accommodated in such a state, only one lower end (entrained portion of the movable pulley 80) of the U shape suffices as the bent portion of the probe cable 3a and thus it is possible to avoid damage to the probe cable 3a.

A cable stopper 78 is provided in each aperture 71b. The cable stopper 78 is a slit 78a narrower than the outside diameter of the probe cable 3a. The probe cable 3a is fitted in an elastically deformed state into the slit 78a and can be retained thereby. In this way the probe cable 3a can be prevented from getting out of the associated aperture 71b or pulled into the cable accommodating space 71a against the operator's will. Thus, when the ultrasonic diagnostic apparatus of this fifth embodiment is used in its fixed state, the probe 3 is held by the cable stopper 78, and the probe holder 8 is not provided.

In each aperture 71b is arranged a roller 79 which can abut the lower surface of the probe cable 3a. With the roller 79, the ingress and egress of the probe cable 3a through the associated aperture 71b are performed smoothly.

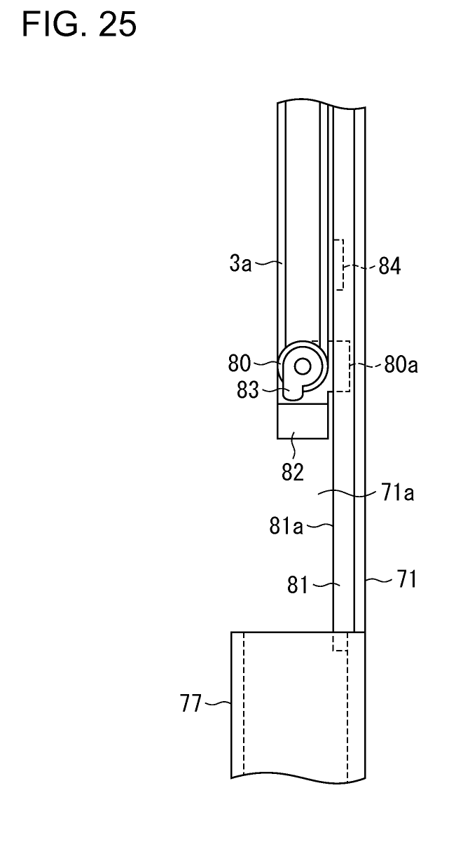

An internal configuration of the cable accommodating device 71 will be described below with reference to FIG. 25. FIG. 25 is an enlarged side view of the movable pulley 80 and the vicinity thereof in a state in which the cover 77 is slid downward and the interior of the cable accommodating device 71 is exposed in its range between approximately 65.0 cm and approximately 100.0 cm in height from the floor surface F (not shown in FIG. 25).

The movable pulley 80, which is, for example, approximately 10.0 millimeters (mm) or more in diameter, is mounted on the U-shaped lower end of the probe cable 3a. With the movable pulley 80, the probe cable 3a can be prevented from being bent at a curvature smaller than the radius (e.g., 5 mm or more) of the movable pulley 80.

The movable pulley 80 is held by a rail 81 vertically movably through a holder 80a. The rail 81 is fixed vertically to a rear surface of the cable accommodating device 71 within the cable accommodating space 71a. The movable pulley 80 is urged downward by a weight 82. The weight 82 serves as a force for pulling the probe cable 3a smoothly into the cable accommodating space 71a.

A cable lock 83 is attached to the movable pulley 80. The cable lock 83 is constituted by a pivotable lever member. With such a cable lock 83, it is possible to prevent disengagement of the probe cable 3a from the movable pulley 80 and hence both safety and reliability are improved.

The rail 81 is provided with a movable pulley lock 84 at a position approximately 70.0 cm high from the floor surface F. The movable pulley lock 84 is a seesaw-like member adapted to move like a seesaw to change the attitude. With the movable pulley lock 84, as will be described later, the movable pulley 80 with the probe cable 3a removed therefrom can be allowed to stop at a position approximately 70.0 cm or more in height from the floor surface F. Thus, the work for mounting or dismounting the probe cable 3a to or from the movable pulley 80 becomes easier to perform.

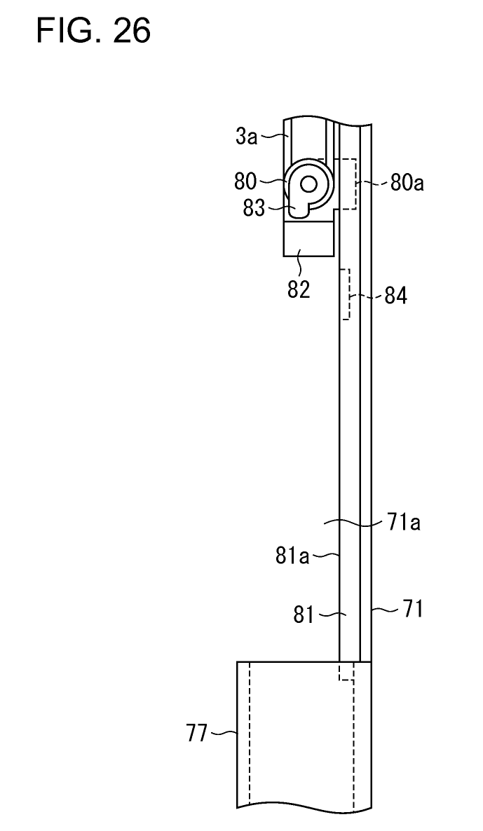

FIGS. 26 to 28 show a procedure for removing the probe cable 3a from the movable pulley 80. For removing the probe cable 3a from the movable pulley 80, first the cover 77 is slid downward, allowing the interior of the cable accommodating device 71 to be exposed in its range between approximately 65.0 cm and approximately 100.0 cm in height from the floor surface F (not shown in FIGS. 26 to 28). In this state the probe cable 3a is pulled out and the movable pulley 80 is raised to a position higher than the movable pulley lock 84 as shown in FIG. 26.

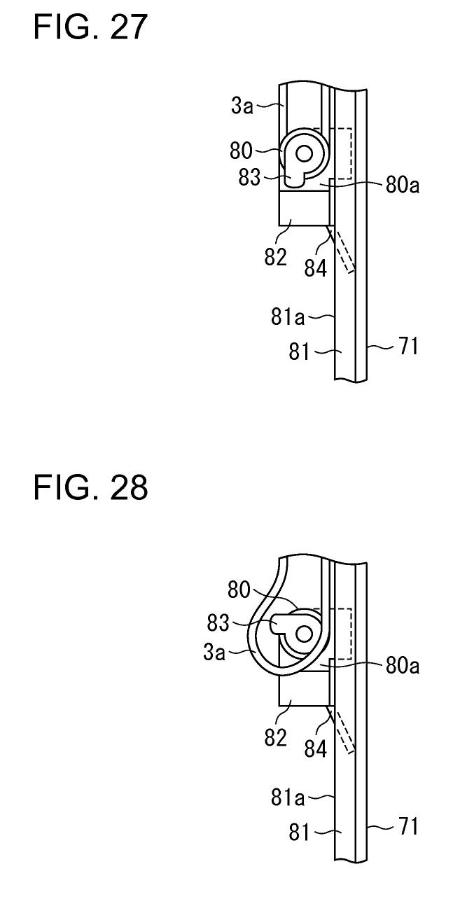

Next, as shown in FIG. 27, the movable pulley lock 84 is moved seesaw-like and is thereby projected to a greater extent than an upper surface 81a of the rail 81, then the movable pulley 80 is brought down and is put on the movable pulley lock 84. Further, as shown in FIG. 28, the cable lock 83 is turned up and the probe cable 3a is removed from the movable pulley 80.

The procedure of mounting the movable pulley 80 onto the probe cable 3a is the reverse to the procedure of removing the probe cable 3a from the movable pulley 80.

When the ultrasonic diagnostic apparatus 70 of this embodiment is to be moved for use, the operating device 2, the image display device 4, the processing device 5, the probe connection unit 7′ and the probe holder 8 are attached to the movable carrier car 100. Though not specially shown, the probe connection unit 7′, unlike the fixed state, may be mounted removably on the processing device 5. Alternatively, also in the movable carrier car 100, the box-like portion 108 of the movable carrier car 100 may be provided at the position permitting the probe connection unit 7′ to be arranged under the processing device 5, and the positional relation between the probe connection unit 7′ and the processing device 5 may be made the same as that in the fixed state.

According to the ultrasonic diagnostic apparatus 70 of this embodiment, the probe connection unit 7′ is mounted to the cable accommodating device 71, the processing device 5 is mounted to the probe connection unit 7′, the image display device 4 is mounted to the support rod 73, and the operating device is mounted to the movable base 10 arranged near the table B, whereby the ultrasonic diagnostic apparatus 70 can be used in its fixed state. On the other hand, the probe connection unit 7′ with the processing device 5 mounted thereto is removed from the cable accommodating device 71, the image display device 4 is removed from the arm 74, further, the operating device 2 is removed from the movable base 10, and they are attached to the movable carrier car 100. The probe holder 8 is also attached to the movable carrier car 100. As a result, the ultrasonic diagnostic apparatus 70 can be moved from the place where it is used in its fixed state to another place for use. Even when the ultrasonic diagnostic apparatus 70 is used in its fixed state, since the image display device 4 is mounted through the arm rotatable in the horizontal direction and capable of extension and retraction in the vertical direction, the image display device 4 can be moved easily to for example a place where the inspector can perform inspection with ease. Thus, the ultrasonic diagnostic apparatus 70 that is employable in a fixed state to a predetermined place can be made more convenient for use.

Besides, since the image display device 4, the processing device 5, the probe connection unit 7′ and the cable accommodating device 71 are arranged on a wall W side opposite to the operating device 2 which must be arranged on the operator side with respect to the table B, the operator-side space becomes wider than in the prior art and it is possible to utilize the space of the installation room R effectively.

In this embodiment, moreover, since the processing device 5 and the probe connection unit 7′ are provided integrally above the cable accommodating device 71 whose portion 100 cm or less in height H from the floor surface F is 10 cm or less in thickness, the gap between the table B and the wall W can be set to for example 11 cm at a position lower than 100 cm in height. Thus, the table B can be approximated as much as possible to the wall W. As a result, the operator-side space becomes still wider than in the prior art and it is possible to utilize the space of the installation room R effectively.

Since the apertures 71b are formed in the cable accommodating device 71 at a position of 120 cm or more in height from the floor surface F which position is sufficiently higher than the subject laid on the table B, it follows that the cable enters and leaves the cable accommodating device 71 from a sufficiently higher position than the subject, whereby the probe cable 3a can be prevented from contacting the subject and giving an unpleasant feeling to the subject.

Although the invention has been described above by way of embodiments thereof, it goes without saying that various changes may be made within the scope not altering the gist of the invention. For example, though not shown specially, the operating device 2 may be of a structure that permits the operating device to be mounted to any of the ceiling C, wall W and floor surface F within the installation room R removably through an arm having movable portions. Further, though not specially shown, the image display device 4 may be of a structure permitting the image display device to be mounted to either the wall W or the floor surface F of the installation room R through an arm having movable portions.

Although the arm 12 with the image display device 4 mounted thereto and the arm 61 with the operating device 2 mounted thereto are attached to the columnar member 9 fixed near the table B, the mounting positions of the arms 12 and 61 need not always be near the table B. The arms 12 and 61 may be made extensible up to near the table B and be fixed to a place somewhat spaced apart from the table B.

As shown in the above first to fourth embodiments, the operating device 2, the image display device 4, the processing device 5, the probe connection unit 7 and the probe holder 8 may be arranged in various layouts. Also in case of using the cable accommodating device 71 without using the probe holder 8 as in the fifth embodiment, the operating device 2, the image display device 4, the processing device 5 and the probe connection unit 7 may be arranged in various layouts. Thus, no limitation is made to the layouts shown in the above embodiments. For example, the operating device 2 and the image display device 4 may be attached to the columnar member 9 removably. Also, the operating device 2, the image display device 4 and the processing device 5 may be attached to the columnar member 9 removably.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
an operating device for an operator to input an instruction;
an image display device configured to display an ultrasonic image; and
a processing device configured to generate the ultrasonic image based on a reception signal resulting from transmission of an ultrasonic wave for display on said image display device,
wherein:
said image display device is mounted removably to one of:
a first end of an arm comprising a movable portion, said arm extending up to near a table for resting a subject thereon, a second end of said arm mounted to any of a ceiling, a wall, and a floor surface of an installation room with the table installed therein; and
an image display device mounting member fixed within the installation room;
said operating device is mounted removably to an operating device mounting member arranged near the table; and
said processing device is mounted removably to a processing device mounting member arranged near the table.

2. An ultrasonic diagnostic apparatus comprising:
an operating device for an operator to input an instruction;
an image display device configured to display an ultrasonic image; and
a processing device configured to generate the ultrasonic image based on of a reception signal resulting from transmission of an ultrasonic wave for display on said image display device,
wherein:
said operating device is mounted removably to one of:
a first end of an arm comprising a movable portion, said arm extending up to near a table for resting a subject thereon, a second end of said arm mounted to any of a ceiling, a wall, and a floor surface of an installation room with the table installed therein; and
an operating device mounting member fixed within the installation room;
said image display device is mounted removably to an image display device mounting member arranged near the table; and
said processing device is mounted removably to a processing device mounting member arranged near the table.

3. An ultrasonic diagnostic apparatus according to claim 1, wherein one of said image display device mounting member, said operating device mounting member, and said processing device mounting member comprises a columnar member capable of extension and retraction, said columnar member is fixed within the installation room by being adjusted to a length of abutment thereof in a pressed state against the ceiling and the floor surface of the installation room.

4. An ultrasonic diagnostic apparatus according to claim 1, wherein at least one of said image display device and said processing device is arranged on a side opposite to a side where said operating device is arranged with respect to the table.

5. An ultrasonic diagnostic apparatus according to claim 1, wherein:
said processing device mounting member comprises a mounting portion comprising a processing device mounting surface perpendicular to the floor surface, said mounting portion further comprising a retention hole and a connector for connection thereto of said processing device being provided so as to be movable in a direction perpendicular to the floor surface;
said processing device comprises a retention lug for engagement in said retention hole on a surface thereof which confronts said processing device mounting surface when mounted to said processing device mounting member; and
said retention hole has a shape and size permitting said retention lug to move in a moving direction of said connector, said retention lug is engaged on a lower portion of said retention hole in an imposed state due to a weight of said processing device.

6. An ultrasonic diagnostic apparatus according to claim 1, further comprising a probe connection unit comprising a probe connector for connection of an ultrasonic wave transmitting and receiving probe is mounted removably to a probe connection unit mounting member arranged near the table.

7. An ultrasonic diagnostic apparatus according to claim 1, further comprising a probe holder configured to hold a probe, said probe holder is mounted removably to a probe holder mounting member arranged near the table.

8. An ultrasonic diagnostic apparatus according to claim 1, further comprising a cable accommodating device for accommodating a cable of a probe.

9. An ultrasonic diagnostic apparatus according to claim 8, wherein said processing device and a probe connection unit are arranged above said cable accommodating device, and in said cable accommodating device with respect to a portion where a height from the floor surface is 70 cm or less is 15 cm or less in thickness.

10. An ultrasonic diagnostic apparatus according to claim 8, wherein a probe connection unit is arranged above said cable accommodating device, said cable accommodating device comprises an aperture in a position 120 cm or more in height from the floor surface, and a cable of a probe is accommodated through said aperture into a cable accommodating space formed within said cable accommodating device.

11. An ultrasonic diagnostic apparatus according to claim 1, wherein said operating device, said image display device, and said processing device are mounted removably onto a movable carrier car.

12. An ultrasonic diagnostic apparatus according to claim 1, wherein one of said arm for mounting said image display device to said image display device mounting member and said arm for mounting said operating device to said operating device mounting member comprises a horizontal arm comprising a plurality of arm members connected rotatably in a horizontal direction, and a vertical arm capable of extension and retraction in a vertical direction.

13. An ultrasonic diagnostic apparatus according to claim 8, wherein said cable accommodating device comprises a cable stopper configured to prevent entry and exit of said cable of said probe to and from a cable accommodating space.

14. An ultrasonic diagnostic apparatus according to claim 8, wherein a roller capable of abutment against a lower surface of said cable of said probe is provided in an aperture formed in said cable accommodating device.

15. An ultrasonic diagnostic apparatus according to claim 10, wherein said cable of said probe is bent in U shape by said aperture and is accommodated in said cable accommodating space.

16. An ultrasonic diagnostic apparatus according to claim 15, wherein a movable pulley is mounted on a lower end of the U shape of said cable accommodated in said cable accommodating space.

17. An ultrasonic diagnostic apparatus according to claim 1, wherein said image display device mounting member and said processing device mounting member are constituted by one and same mounting member.

18. An ultrasonic diagnostic apparatus according to claim 1, wherein said image display device mounting member and said operating device mounting member are constituted by one and same mounting member.

19. An ultrasonic diagnostic apparatus according to claim 1, wherein said operating device mounting member and said processing device mounting member are constituted by one and same mounting member.

20. An ultrasonic diagnostic apparatus according to claim 1, wherein said image display device mounting member, said operating device mounting member, and said processing device mounting member are constituted by one and same mounting member.
